(19) United States
(12) Patent Application Publication
(10) Pub. No.: US 2007/0266774 A1
(43) Pub. Date: Nov. 22, 2007

(54) METHOD FOR EVALUATING ASTHMA CONTROL

(76) Inventors: Peter Gerard Gibson, Merewether (AU); Lisa Gai Wood, Merewether (AU); Manohar Lal Garg, Wallsend (AU)

Correspondence Address: QUARLES & BRADY LLP, 33 E. MAIN ST, SUITE 900, P.O BOX 2113, MADISON, WI, 53701-2113, US

(21) Appl. No.: 11/562,741

(22) Filed: Nov. 22, 2006

(30) Foreign Application Priority Data
May 16, 2006 (AU) AU 2006902620

(51) Int. Cl. G01N 33/48

(52) U.S. Cl. 73/53.01

(57) ABSTRACT

Methods for diagnosing, monitoring and evaluating asthma control in subjects and methods of monitoring the efficacy of asthma treatment regimes in said subjects using blood levels of α-tocopherol quinone.

### FIELD OF THE INVENTION

The present invention relates generally to methods for monitoring and evaluating asthma control in subjects and thus to methods of monitoring the efficacy of asthma treatment regimes in said subjects. Methods of the invention also find application in the diagnosis of asthma.

### BACKGROUND OF THE INVENTION

Asthma is a chronic inflammatory disorder of the airways, involving variable airflow obstruction and increased airway responsiveness to a variety of stimuli. These stimuli may lead to the respiratory burst of activated inflammatory cells, resulting in the production of excessive quantities of free radicals, which overwhelm host antioxidant defences leading to oxidative stress.

Host defence against oxidative stress is provided both by exogenous antioxidants, typically obtained from dietary sources, and by endogenous antioxidants manufactured within the body. Antioxidant defences typically present include superoxide dismutase, glutathione peroxidise (GSHPx), glutathione reductase, catalase, β-carotene, α-carotene, ascorbic acid (vitamin C) and α-tocopherol (vitamin E).

Disturbed antioxidant levels have been identified in asthma^{[1]} with deficiencies in the exogenous antioxidants vitamin C, vitamin E^{[2,4-6]}, β-carotene and α-carotene having been reported. Deficiencies in the endogenous antioxidants superoxide dismutase and its cofactor Zn, glutathione peroxidase and its co-factor Se, catalase and glutathione (GSH)^{[3,5,7]} have also been reported. However studies to date have been inconclusive in establishing meaningful correlations between antioxidant levels and asthma, and indeed in some cases studies have revealed contradictory outcomes.

With the increasing prevalence of asthma worldwide, international guidelines for the diagnosis, evaluation, monitoring and treatment of asthma have been established. According to guidelines issued by the World Health Organisation and the National Institute of Health's Global Initiative for Asthma (GINA) (see www.ginasthma.com), a primary goal of asthma treatment is to achieve effective asthma control, and in particular the minimisation of symptoms and exacerbations with the least use of medication. Asthma control relates to the adequacy of asthma treatment, and as defined by Juniper et al. (1999)^{[8]}, describes the full range of clinical impairment that asthma sufferers experience. The range is typically from well controlled, in which the patient is completely unimpaired and unlimited, to extremely poorly controlled which may be life-threatening^{[8]}.

The effective monitoring of patient status and the efficacy of any treatment regimes that patients may be receiving is a constant challenge. Current methods include survey and questionnaire based determinations. However such methods are largely based on self-assessment and self-reporting of symptoms by patients and hence their reliability depends to an extent on compliance by patients. There is a need for more reliable and more objective means of monitoring and evaluating asthma control.

Currently, blood serum levels of eosinophilic cationic protein (ECP) are measured to monitor asthma control. However, less than 50% of symptomatic asthmatics experience eosinophilic inflammation making serum ECP measurements irrelevant in the majority of patients.

Accordingly, there remains a clear need for improved methods and tests for reliable monitoring of asthma control and of the efficacy of asthma treatment which are more widely applicable to asthmatic patients.

### SUMMARY OF THE INVENTION

The present invention is predicated on the inventors' surprising findings that blood levels of α-tocopherol quinone (an oxidised derivative of α-tocopherol) and the ratio of α-tocopherol quinone to α-tocopherol are elevated in asthmatics as compared to non-asthmatics. Further, the ratio of α-tocopherol quinone to α-tocopherol in whole blood correlates with the status of asthma control in asthmatics.

Accordingly, a first aspect of the present invention provides a method for assessing asthma control in a subject, the method comprising:

(a) obtaining a biological sample from the subject comprising non-lung derived body fluid and/or tissue; and

(b) determining the level of at least one oxidised form of vitamin E or a derivative or metabolite thereof in the non-lung body fluid and/or tissue,

wherein the level of the at least one oxidised form of vitamin E or derivative or metabolite thereof is indicative of the asthma control in the subject.

The non-lung body fluid and/or tissue present in the sample may be any such body fluid or tissue in which the level of an oxidised form of vitamin E or derivative or metabolite thereof correlates with the asthma control in the subject. For example, the fluid or tissue may be selected from urine, saliva, whole blood, blood plasma, and blood serum. Typically, the body fluid or tissue is peripheral whole blood.

The oxidised form(s) of vitamin E may comprise any of the oxidised forms of the tocopherols or tocotrienols in the vitamin E family, such as α-tocopherol quinone and 2,3- and 5,6-epoxy-alpha-tocopherylquinones. Typically, the oxidised form of vitamin E is α-tocopherol quinone, an oxidation product of α-tocopherol.

The method may also comprise comparing the level of at least one oxidised form of vitamin E or derivative or metabolite thereof in the sample obtained from the subject with the level of at least one oxidised form of vitamin E or derivative or metabolite thereof from one or more control samples. Typically a control sample is a sample from a subject known to have normal levels of the oxidised compound and/or a subject known not to be asthmatic.

The method may further comprise determining the level of vitamin E in the non-lung body fluid and/or tissue and calculating the ratio of oxidised vitamin E to vitamin E, wherein the ratio is indicative of the asthma control in the subject. The method may also include comparing the ratio in the sample obtained from the subject with the ratio from one or more control samples. Typically a control sample is a sample from a subject known to have normal levels of the oxidised compound and/or a subject known not to be asthmatic.

The vitamin E analysed may be one or more of the reduced forms of the tocopherols and/or tocotrienols of the vitamin E family. Typically the vitamin E analysed is α-tocopherol.

The method may also comprise assessing asthma control in the subject by one or more alternative means, such as the Asthma Control Questionnaire. The data so obtained may be correlated with the level of the at least one oxidised form of vitamin E or derivative or metabolite thereof and/or with the ratio of ratio of oxidised vitamin E to vitamin E.

A second aspect of the present invention provides a method for determining asthma control in a subject, the method comprising:

(a) obtaining a biological sample from the subject comprising non-lung derived body fluid and/or tissue;

(b) determining the level of at least one oxidised form of vitamin E or a derivative or metabolite thereof in non-lung body fluid and/or tissue;

(c) determining the level of vitamin E in the non-lung body fluid and/or tissue; and

(d) calculating the ratio of the level determined in (b) to that determined in (c),

wherein the ratio is indicative of the asthma control in the subject.

According to a third aspect of the present invention there is provided a method for monitoring asthma control in a subject, the method comprising:

(a) obtaining a biological sample from the subject comprising non-lung derived body fluid and/or tissue;

(b) determining the level of at least one oxidised form of vitamin E or a derivative or metabolite thereof in the non-lung body fluid and/or tissue;

(c) repeating steps (a) and (b) at least once over a period of time; and

(d) determining whether the level has changed over the period of time,

wherein a change in the level is indicative of a change in asthma control in the subject.

The method may also comprise determining the level of vitamin E in the non-lung body fluid and/or tissue more than once over the period of time and calculating the ratio of oxidised vitamin E to vitamin E, wherein a change in the ratio is indicative of a change in asthma control in the subject.

According to a fourth aspect of the present invention there is provided a method for determining an appropriate asthma treatment regime for a subject, the method comprising:

(a) obtaining a biological sample from the subject comprising non-lung derived body fluid and/or tissue;

(b) determining the level of at least one oxidised form of vitamin E or a derivative or metabolite thereof in the non-lung body fluid and/or tissue;

(c) evaluating the asthma control in the subject on the basis of the level determined in (b); and

(d) selecting a treatment regime for the subject on the basis of the evaluated asthma control.

The method may also comprise determining the level of vitamin E in the non-lung body fluid and/or tissue, calculating the ratio of oxidised vitamin E to vitamin E and selecting the treatment regime on the basis of the ratio.

According to a fifth aspect of the present invention there is provided a method for evaluating the efficacy of an asthma treatment regime in a subject, the method comprising:

(a) treating the subject with a treatment regime for a period sufficient to evaluate the efficacy of the regime;

(b) obtaining a biological sample from the subject comprising non-lung derived body fluid and/or tissue;

(c) determining the level of at least one oxidised form of vitamin E or a derivative or metabolite thereof in the non-lung body fluid and/or tissue;

(d) repeating steps (b) and (c) at least once over a period of time; and

(e) determining whether the level has changed over the period of time,

wherein a change in the level is indicative of a change in asthma control in the subject and the degree of efficacy of the treatment regime.

A decrease in the level of the oxidised form of the vitamin or the derivative thereof is indicative that the treatment is effective in treating the asthma, with a greater decrease indicating greater effectiveness of the treatment. Similarly, improved asthma control may be determined by a sustained reduction in the level of the oxidised form of vitamin E or the derivative thereof in the body fluid over the period of time.

The method may also comprise determining the level of vitamin E in the non-lung body fluid and/or tissue more than once over the period of time and calculating the ratio of oxidised vitamin E to vitamin E, wherein a change in the ratio is indicative of a change in asthma control and the degree of efficacy of the treatment regime.

According to a sixth aspect of the present invention there is provided a method for diagnosing asthma in a subject, the method comprising:

(a) obtaining a biological sample from the subject comprising non-lung derived body fluid and/or tissue; and

(b) determining the level of at least one oxidised form of vitamin E or a derivative or metabolite thereof in the non-lung body fluid and/or tissue,

wherein the level of the at least one oxidised form of vitamin E or derivative or metabolite thereof is indicative of asthma in the subject.

According to a seventh aspect of the present invention there is provided a method for diagnosing asthma in a subject, the method comprising:

(a) obtaining a biological sample from the subject comprising non-lung derived body fluid and/or tissue;

(b) determining the level of at least one oxidised form of vitamin E or a derivative or metabolite thereof in non-lung body fluid and/or tissue;

(c) determining the level of vitamin E in the non-lung body fluid and/or tissue; and

(d) calculating the ratio of the level determined in (b) to that determined in (c),

wherein the ratio is indicative of asthma in the subject.

Methods embodied by the invention are particularly suitable for evaluating the status of asthma control in human subjects. However, the invention is not limited thereto and extends to any mammal useful as a model for asthma in humans. Typically the subject is a mammal, more typically a human.

The features and advantages of methods of the present invention will become further apparent from the following detailed description of embodiments.

### BRIEF DESCRIPTION OF THE DRAWINGS

The invention will now be described by way of example only, with reference to the accompanying drawings.

FIG. 1: Graph showing sputum supernatant concentrations of total glutathione, reduced glutathione, oxidised glutathione (GSSG) and % oxidized glutathione (% GSSG) in asthma versus healthy controls (^{a}p<0.005 versus controls; ^{b}p<0.05 versus controls).

FIG. 2: Graph showing sputum supernatant concentrations of oxidised glutathione versus FEV₁/FVC % (r=−0.316, p=0.029). Data analysed using Spearman's rank correlation.

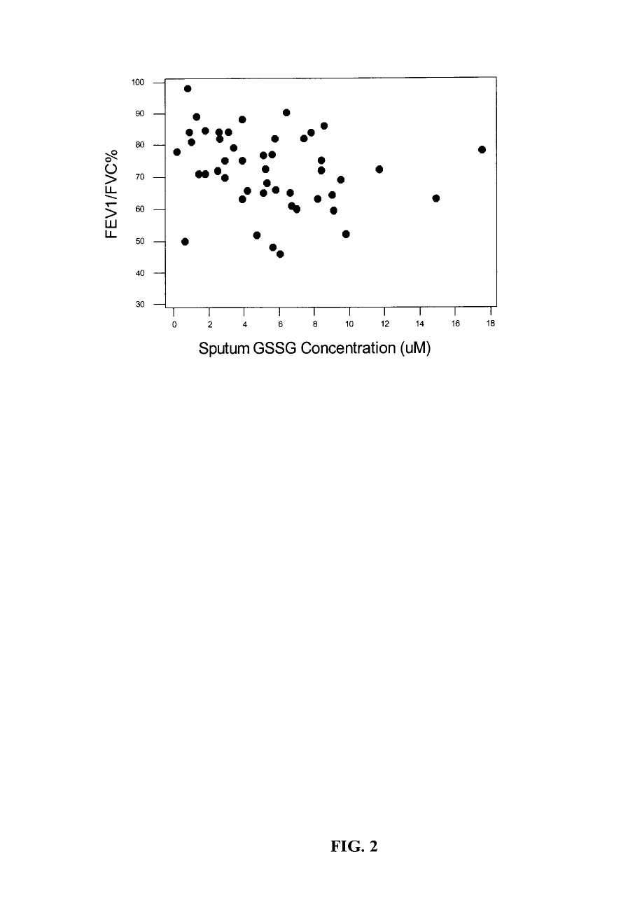

FIG. 3: Graph showing whole blood concentrations of α-tocopherol, α-tocopherol quinone and % α-tocopherol quinone in asthma versus healthy controls (^{a}p=0.076 versus controls; ^{b}p<0.05 versus controls;).

FIG. 4: Graph showing % α-tocopherol quinone in whole blood versus asthma control score (r=0.804, p=0.009). Data analysed using Spearman's rank correlation.

### DETAILED DESCRIPTION OF THE INVENTION

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

As used herein the terms “vitamin E” and “tocopherol” are used interchangeably. Vitamin E comprises a family of at least 8 structurally related molecules, the tocopherols and tocotrienols, including α-tocopherol, β-tocopherol, γ-tocopherol, δ-tocopherol, α-tocotrienol, β-tocotrienenol, γ-tocotrienol and δ-tocotrienol.

The term “derivative or metabolite thereof” as used herein in relation to an oxidised form of vitamin E includes within its scope any derivative or metabolite of the oxidised compound with the exception of a reduced form thereof.

As used herein, the term “asthma control” means the status of the disease, typically in light of intervention to treat the disease. Thus asthma control describes the range of symptoms and conditions experienced and suffered by asthmatic patients as a result of their asthma. Asthma control effectively provides a measure at a given point in time of the disease status of an individual, reflecting both current therapeutic treatment regimes used by the individual and the individual's recent exposure to triggers. Several approaches exist for measuring and quantifying asthma control, such quantification providing an asthma control ‘score’. Typically the lower the score, the better the level of asthma control.

As disclosed herein, the inventors have found that levels of α-tocopherol quinone and the ratio of α-tocopherol quinone to α-tocopherol (% α-tocopherol quinone) correlate with asthma control. Higher % α-tocopherol quinone (i.e. higher ratio of α-tocopherol quinone to α-tocopherol) has surprisingly been found to correlate with poorer levels or status of asthma control in asthmatic patients (higher asthma control score as determined by the Asthma Control Questionnaire^{[8]} as exemplified herein).

Thus, disclosed herein for the first time is a simple biochemical test applicable to asthmatics of all inflammatory phenotypes (eosinophilic and neutrophilic), that facilitates the assessment and monitoring of asthma control, the identification of appropriate therapeutic treatment regimes for individual patients, and assess and monitor the effectiveness of existing treatments.

Accordingly, an aspect the invention provides a method for assessing asthma control in a subject, the method comprising: obtaining a biological sample from the subject comprising non-lung derived body fluid and/or tissue; and determining the level of at least one oxidised form of vitamin E or a derivative or metabolite thereof in the non-lung body fluid and/or tissue, wherein the level of the at least one oxidised form of vitamin E or derivative or metabolite thereof is indicative of the asthma control in the subject.

Further, based on the surprising finding described herein that blood levels of α-tocopherol quinone are elevated in asthmatics as compared to non-asthmatics, the present invention also finds application in diagnosing asthma in individuals, for example in those who may present with one or more symptoms of asthma but in whom asthma has not been confirmed. Embodiments of the invention may therefore be used alone or more typically in conjunction with or as an adjunct to one or more other diagnostic methods and tests to determine the likelihood that an individual may suffer from asthma or to diagnose the disease. Such other diagnostic methods and tests will be well known to those skilled in the art.

The levels of oxidised compound(s) determined according to methods of the invention can be compared to control values as a suitable reference to assist in determining the level or status of asthma control in subjects. For example, levels of (x-tocopherol quinone or other oxidised forms of vitamin E, or derivatives or metabolites thereof, may be determined in one or more, typically a population, of non-asthmatic individuals. Alternatively or in addition, levels of the oxidised compound(s) may be determined in a range of individuals with different levels of asthma control, each reference or control level typically being correlated with a predetermined asthma control score. In subjects to which methods of the invention are applied, levels of the oxidised compound(s) lower than the control values may be indicative of satisfactory asthma control, whilst measured levels higher than the control values may be indicative of poorer or unsatisfactory asthma control.

Methods of the invention may also comprise determining whether the asthma control score for a subject is within a predetermined range indicative of satisfactory asthma control. An asthma control score outside of the predetermined range can be used to indicate that the subject's asthma treatment needs to be modified to improve asthma control in the subject or that the subject should otherwise be placed on a suitable treatment regime to improve asthma control. The analysis may be repeated one or more times over a given period of time to monitor asthma control in the subject over time. Determination of the level or status of asthma control in a subject, in particular the monitoring of asthma control over time, also facilitates decision making with respect to the most appropriate intervention or treatment regime for an individual subject. The treatment regime will typically be tailored so as to obtain a sustained reduction in the level of oxidised vitamin E in the body fluid of the subject. For example, this may comprise introducing a new treatment regime or modifying an existing regime with a view to improving asthma control in the subject. The modification of a regime may be modification with respect to any one or more of a variety of factors, such as the nature of any anti-asthma medication, the dosage thereof, the timing of administration and/or any supplementary asthma management strategies. In the event that a subject's asthma control is good and is maintained at a sufficient level for a suitable period of time, the subject may be removed from treatment. Such decision making with respect to treatment regimes will vary from case to case and the determination of the most appropriate strategy is well within the expertise and experience of those skilled in the art.

In further embodiments of the invention, a ratio of the level of the oxidised form of vitamin E, or the derivative or metabolite thereof, to the reduced form of the vitamin can be utilised. Thus, the present invention also provides methods for determining asthma control or diagnosing asthma in a subject, comprising: obtaining a biological sample from the subject comprising non-lung derived body fluid and/or tissue; determining the level of at least one oxidised form of vitamin E or a derivative or metabolite thereof in non-lung body fluid and/or tissue; determining the level of vitamin E in the non-lung body fluid and/or tissue; and calculating the ratio of the levels of oxidised vitamin E to vitamin E.

According to the invention, data obtained in accordance with methods disclosed herein may be correlated with asthma control scores as determined by one or more alternative means, for example using survey/questionnaire-based methods to quantitate asthma control. Such survey/questionnaire-based methods typically involve the evaluation of parameter(s) normally associated with asthma such as asthma symptoms, severity of the condition, peak lung flow volumes, asthma medications utilised and dosages administered, and frequency of use of asthma medication. Asthma control scores can for instance be determined utilising the validated Asthma Control Questionnaire (ACQ) of Juniper et al. (1999)^{[8]}. The ACQ includes seven questions, six of which are completed by the subject based on their symptom experiences of the previous 7 days, while the final question relates to FEV1 values and is completed by the relevant clinician. All questions are responded to using a seven point scale. All questions are given equal weighting and the ACQ asthma control score is the mean of the seven responses, from 0 (well controlled) to 6 (extremely poorly controlled). An alternative questionnaire also allowing a quantitative measure of asthma control, the Asthma Control Scoring System (ACSS) has also been developed^{[9]}.

Those skilled in the art will readily appreciate that vitamin E and the oxidised form of the vitamin, or the derivatives or metabolites thereof, may be measured utilising any suitable assay protocols known in the art such as, for example, high performance liquid chromatography (HPLC) or gas chromatography/mass spectrometry (GC-MS). Those skilled in the art will appreciate that the present invention is not limited by reference to the means by which the compound(s) are detected or measured.

Derivatives and metabolites of oxidised forms of vitamin E as contemplated by the invention include natural metabolites, degradation products and artificial derivatives as may derived, for example, from chemical or other treatments involved in the assay protocols employed. Such artificial derivatives include oxidised forms of the vitamin with modified substituent group(s) or chemical bond(s), or which have been modified in some other way such as by the addition of side chain(s) or by being coupled to a reporter group or another compound for its detection. Natural metabolites and degradation products of oxidised forms of tocopherols in the vitamin E family include tocopheryl hydroquinone, tocopheronic acid and tocopheronolactone.

A treatment regime for the treatment of asthma in a subject in accordance with a method of the invention may involve administration of any of the asthma medications commonly utilised in the treatment of asthma including but not limited to, β₂ agonists, and oral and inhaled steroids. Such steroids include drugs known as asthma “relievers” such as salbutamol, terbutaline, ipratropium bromide, theophylline, asthma “preventers” such as beclomethasone dipropionate, budesonide and fluticasone, and asthma “controllers” such as salmeterol, eformoterol and theophylline (slow release). Those skilled in the art will appreciate that the invention is not limited by the particular treatment regime employed.

The treatment regime may comprise the administration of a number of these drugs simultaneously, sequentially, or in combination with each other or with non-drug treatments. The type of drug(s) administered, dosage, and the frequency of administration can be determined by medical physicians in accordance with accepted medical principles, and will depend on factors such as the severity of the asthma, the age and weight of the subject, the medical history of the subject, other medication being taken by the subject, existing ailments and any other health related factors normally considered when determining treatments for asthma.

All the essential components required for analysing either or both of the oxidised and reduced forms of Vitamin E, or metabolites or derivatives thereof, in samples of non-lung body fluids and/or tissue in accordance with methods of the present invention may be assembled together in a kit. The kits may optionally include appropriate components for measuring and/or quanititing levels of the vitamin, appropriate positive and negative controls, dilution buffers and the like. In some embodiments, the kits comprise instructions for performing the methods of the present invention.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The present invention will now be described with reference to the following specific examples, which should not be construed as in any way limiting the scope of the invention.

### EXAMPLES

### Example 1

### Determination of Relative Concentrations of Reduced and Oxidised Glutathione and α-Tocopherol in the Airways in Sputum and in Peripheral Blood

The exogenous antioxidant vitamin E exists in vivo primarily in the form of α-tocopherol and is typically present in the respiratory tract. Glutathione and α-tocopherol may protect against airway oxidative damage. Glutathione (GSH) is an endogenous antioxidant that plays a prominent role in the respiratory tract. α-tocopherol is oxidised to α-tocopherol quinone. Glutathione disulphide (GSSG) is a stable oxidised form of glutathione and can be readily measured by a colorimetric assay.

The reduced and oxidized forms of these antioxidants were examined in asthmatic patients and in healthy non-asthmatic individuals using induced sputum and compared to systemic levels in peripheral blood.

### Subjects

Adults (over 18 years) with current diagnosis of asthma were recruited from specialist clinics at John Hunter Hospital, Newcastle, Australia. Controls were recruited by advertisement and asthma was excluded on the basis of history, normal spirometry and airway responsiveness, together with data review by a specialist medical practitioner. Current smokers were excluded. Both stable asthmatics (n=44) and healthy (control) individuals (n=31) were recruited. There was no significant difference in mean age between the two groups.

### Clinical Classification of Asthma

Asthma was diagnosed based upon a history of current (previous 12 months) episodic respiratory symptoms, a prior doctor's diagnosis of asthma (ever), the current (previous 12 months) use of inhaled asthma therapy (β₂-agonists, corticosteroids or cromolyn) and airway hyperresponsiveness following hypertonic saline challenge (% fall in predicted forced expiratory volume in one second (FEV₁)>15%). Subjects were considered to be unstable, and thus excluded from the study, if their asthma had worsened such that they had needed to: attend their doctor or hospital, increase the use of their asthma medication (β₂-agonist, inhaled or oral steroid), or reduce their activity in the previous 4 weeks. Asthma control was measured by a validated questionnaire, the Asthma Control Questionnairer^{[8]} that scores symptoms, activity level, bronchodilator use and lung function during the previous week. According to the Asthma Control Questionnaire, asthma control scores ranges from 0 and 6, and a higher score indicates worse asthma control.

### Sputum Induction and Analysis

Spirometry (KoKo K313100 PD Instrumentation, Louisville, Colo., USA) and combined bronchial provocation and sputum induction with hypertonic saline (4.5%) were performed as described by Gibson et al^{[10]}. Sputum induction time was standardized at 15.5 minutes. A portion of sputum was selected from saliva, dispersed with dithiothreitol (DTT), and a total cell count of leucocytes and viability performed. Cytospins were prepared, stained (May-Grunwald Geimsa) and a differential cell count obtained from 400 non-squamous cells.

### Glutathione (Total and Oxidised) Measurements

Preparation for GSHt: Sputum was selected from saliva, combined with 2 volumes of chilled PBS pH and vortex mixed. Cell free supernatant was obtained by centrifugation (50000 g, 4° C. for 30 min) and stored at −80° C. Whole blood collected in EDTA tubes and selected sputum plugs were also stored at −80° C.

Preparation for GSSG: Sputum supernatant: Sputum was selected from saliva, combined with 10 mM 1-methyl-2-vinylpyridinium trifluoromethanesulfonate (M2VP) and 2 volumes of chilled PBS and vortexed. Cell free supernatant was obtained by centrifugation (50 000 g, 4° C. for 30 minutes) and stored at −80° C. Whole blood: Whole blood was mixed with 10 mM M2VP and stored at −80° C. Whole sputum: Sputum was selected from saliva, vortex mixed with 10 mM M2VP, then stored at −80° C.

Glutathione assay: Glutathione measurements in sputum supernatant and whole blood were performed within 30 days of sample collection. On the day of assay, whole blood samples (for both GSHt and GSSG analysis) were thawed, 5% metaphosphoric acid added, vortex mixed and then centrifuged (1000 g, 4° C., 10 minutes) prior to assay of the supernatant. Whole sputum plugs (for both GSHt and GSSG analysis) were thawed, sonicated using a probe sonicator, and centrifuged (50 000 g, 4° C. for 30 minutes) prior to assay of the supernatant. Concentrations of GSHt and GSSG were determined by colorimetric assay (Oxis International Inc., Portland, Oreg., USA) with standard curves based on dilutions of purified GSSG. Reduced glutathione (GSHr) concentrations were calculated from GSHt and GSSG data, using the formula GSHr=GSHt−2×GSSG. The limit of detection of GSSG was 0.54 μM. This assay was validated for use in sputum supernatant according to guidelines published by the European Respiratory Society for the analysis of fluid phase mediators in induced sputumr^{[11]}. Spiking experiments yielded excellent recovery of the spiked mediator. The average recovery rate for GSSG in sputum supernatant was 103 (range 97-111) %. Within patient reproducibility was good. Subjects (n=5) studied on 2 occasions, 2 days apart, showed all values lie within Bland-Altman limits of agreement (mean bias±2SD) of −20.7 to 10.1.

### α-Tocopherol (Reduced and Oxidized) Measurements

Plasma was separated from whole blood by centrifugation (4° C., 3000 g, 10 min) and stored at −80° C. Mucus plugs were selected from induced sputum samples and stored at −80° C. The concentrations of (α-tocopherol and (α-tocopherol quinone in whole blood, plasma and induced sputum were determined using HPLC. Samples were analysed using an extraction method previously used for plasma samples (see Barua et al, 1993)^{[12]}. Briefly, samples were deproteinated with ethanol, and an equal volume of ethyl acetate (1:1) containing internal standards (canthaxanthin and tocopherol acetate) was then added to the sample. The solution was sonicated using a probe sonicator and centrifuged (3000 g, 4° C. for 1 minute) and the supernatant collected. Ethyl acetate was added to the remaining pellet, the mixture was sonicated, centrifuged (3000 g, 4° C. for 1 minute) and the supernatant was collected. This process was repeated. Hexane was subsequently added to the pellet, the mixture was sonicated, centrifuged and again, the supernatant was collected. Finally ultra pure water was added to the combined supernatants, the mixture was sonicated and centrifuged. The supernatant was decanted, the solvents evaporated with nitrogen and the sample reconstituted in dichloromethane-methanol (1:2 v/v). Chromatography was performed on a Hypersil ODS column (100 mm×2.1 mm×5 um). The mobile phase consisted of acetonitrile:dichloromethane:methanol 0.05% ammonium acetate (85:10:5 v/v) at a flow rate of 0.3 mL/min. Tocopherols and tocopherol quinone were detected at 290 and 260 nm respectively using a photodiode array detector. % α-tocopherol quinone was calculated using the formula % α-tocopherol quinone=α-tocopherol quinone/(α-tocopherol+α-tocopherol quinone)×100.

### Statistical Analysis

Results were analysed using Minitab version 13.32 for Windows (Minitab Inc., State college, Pa., USA). Data was tested for normality using the Anderson-Darling test. Statistical comparisons were performed using the Student t-test for normally distributed data, and the Mann-Whitney U test for non-parametric data. The mean±standard error is reported for normal data, for non-parametric data the median (quartile 1-3) are reported. Group comparisons were conducted using analysis of variance with ANOVA testing for normally distributed variables and Kruskal-Wallis testing for non-parametric data.

Associations between variables were examined using Pearson's correlation coefficient for normally distributed data, and Spearman's rank correlation coefficient for non-parametric data. Significance was accepted if p<0.05.

### Characterization of Subjects

Characteristics of the stable asthmatic (n=44) subjects and healthy control (n=31) subjects are described in Table 1. As expected, the asthmatic subjects had reduced lung function and increased sputum eosinophils compared to controls (see Table 1 and 2).

TABLE 1
Characteristics of stable asthmatics and healthy controls

| | | Healthy |
|---|---|---|
| | Asthma | Controls |
| n | 44 | 31 |
| age (years) | 47.4 ± 2.6 | 41.4 ± 2.5 |
| sex (M/F) | 16/28 | 17/14 |
| % predicted FEV₁ | 82.7 ± 3.4* | 101.4 ± 2.3 |
| % predicted FVC | 98.1 ± 2.6 | 104.1 ± 1.9 |
| % FEV₁/FVC | 68.8 ± 1.8* | 80.5 ± 1.3 |
| PD15 (mLs)^{a,b} | 0.65 (0.35) | NA |
| Atopy n (%) | 41 (93%) | 15 (58%) |
| Asthma Control Questionnaire | 1.4 ± 0.1 | NA |
| Inhaled corticosteroid use (μg/day) | 1000 (500–1600) | NA |

NA = not applicable
*p < 0.001 versus healthy controls
Data are normally distributed and presented as mean ± SEM
Data presented as median (quartile 1–quartile 3)
^{a}PD15 values are geometric mean (log SD);
^{b}n = 26

TABLE 2
Induced Sputum Inflammatory Cell Counts

| | | Healthy |
|---|---|---|
| | Asthma | Controls |
| Total cell count (×10⁶/mL) | 1.8 | 1.7 |
| | (1.4–3.9) | (1.0–2.9) |
| % Neutrophils | 25.5 | 24.3 |
| | (15.7–44.9) | (12.1–47.4) |
| % Eosinophils | 1.5* | 0.0 |
| | (0.4–5.4) | (0.0–1.0) |
| % Macrophages | 56.8 | 66.3 |
| | (33.0–76.1) | (41.9–82.8) |
| % Lymphocytes | 0.5 | 0.94 |
| | (0.0–1.1) | (0.1–2.0) |
| % Columnar epithelial cells | 2.0 | 0.75 |
| | (0.8–5.2) | (0.3–2.8) |
| % Squamous cells | 3.2 | 6.1 |
| | (0.6–11.4) | (1.1–17.5) |

Data presented as median (quartile 1–quartile 3)
*p = 0.005 versus healthy controls.

### Glutathione

Concentrations of GSHt and GSSG in whole blood, plasma, whole sputum and sputum supernatant are shown in Table 3.

In peripheral whole blood, GSHt was predominantly intracellular, with only a small proportion existing as GSSG. The concentration of GSHt in sputum supernatant was high relative to plasma (12.0 (6.3-18.2) versus 0.44 (0.39-0.95) uM respectively, p<0.0001) (see Table 3). The proportion of oxidised glutathione was high in sputum (whole and supernatant) compared to whole blood.

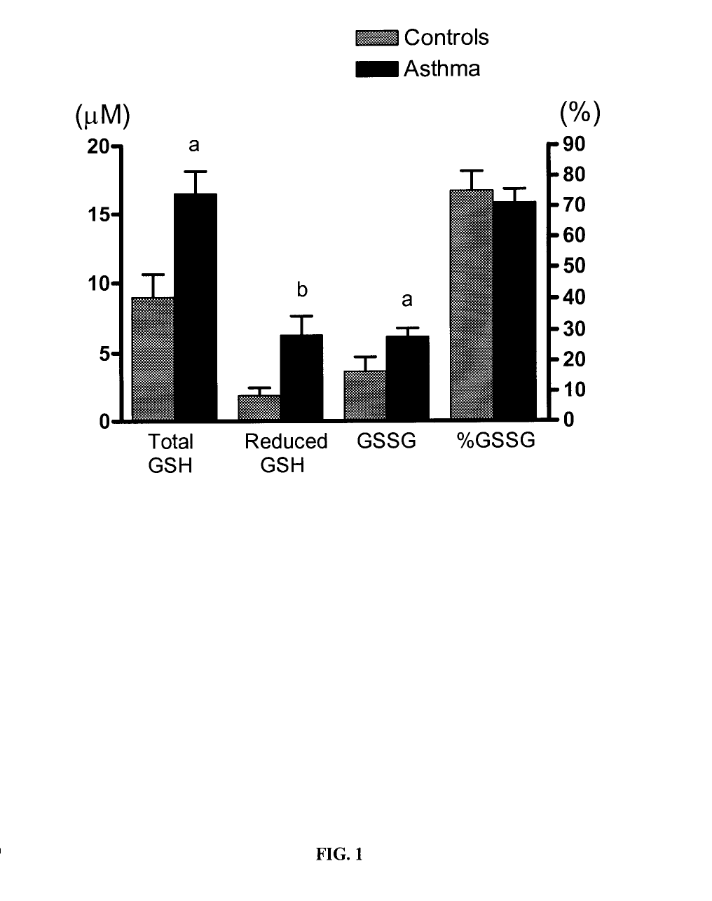

Levels of GSHt, GSHr and GSSG in sputum supernatant were elevated in asthma versus controls (GSHt; 15.3 (10.0-22.4) versus 7.0 (4.7-14.3) μM, p=0.002, GSHr; 4.1 (1.4-6.8) versus 1.2 (0.0-3.8) μM, p=0.026 and GSSG; 5.9 (4.0-8.4) versus 2.6 (1.8-5.1) μM, p=0.005) (see Table 3, FIG. 1).

### α-Tocopherol

Concentrations of α-tocopherol and α-tocopherol quinone in whole blood, plasma and whole sputum are shown in Table 4. Whole sputum levels of α-tocopherol were similar to whole blood levels. The proportion of α-tocopherol quinone was much higher in whole blood than plasma or sputum.

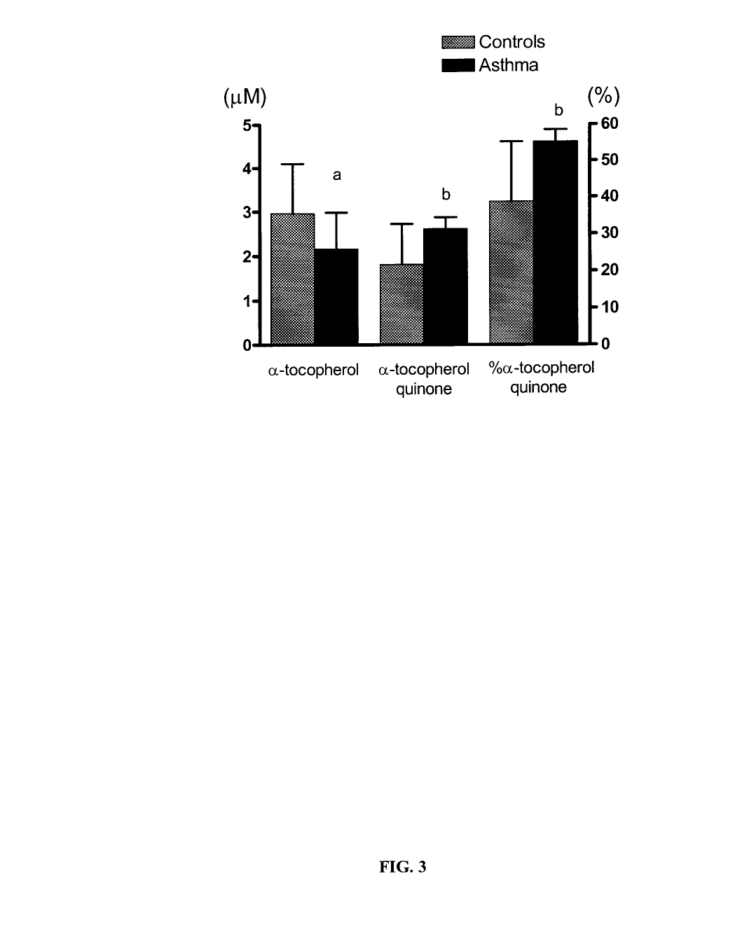

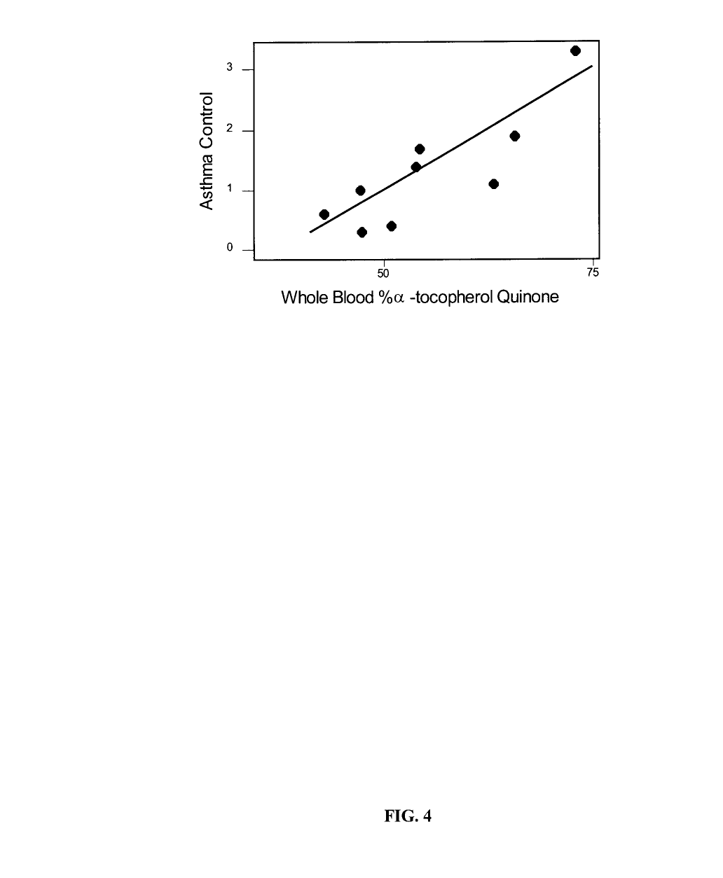

Whole blood levels of α-tocopherol were low in asthma versus controls (2.2 (1.5-2.8) versus 2.8 (2.1-3.7) mg/L, p=0.076)(see FIG. 3). Similarly, plasma α-tocopherol levels were also low in asthma compared to controls (7.3 (5.7-8.1) versus 12.5 (6.6-18.6) mg/L respectively, p=0.020] (see Table 4). Whole blood levels of α-tocopherol quinone and % α-tocopherol quinone were elevated in asthma versus controls (α-tocopherol quinone; 2.4 (2.1-3.3) mg/L versus 1.6 (1.0-2.5) mg/L, p=0.039 and α-tocopherol quinone; 53.8 (47.2-64.4) % versus 44.6 (21.0-51.9) %, p=0.039)(see FIG. 3). There were no differences between asthmatics and healthy controls in whole sputum α-tocopherol or α-tocopherol quinone levels. % α-tocopherol quinone in whole blood correlated with asthma control (r=0.804, p=0.009) (see FIG. 4). Sputum concentrations of α-tocopherol quinone correlated with sputum supernatant concentrations of GSSG (r=0.608, p=0.047). There was no relationship between α-tocopherol and sputum cell counts.

TABLE 3
Systemic (peripheral blood) and airway (induced sputum)
levels of total and oxidized glutathione

| | N (Asthma/ | GSHt (μM) | | GSHr (μM) | | GSSG (μM) | |
|---|---|---|---|---|---|---|---|
| | Control) | Asthma | Controls | Asthma | Controls | Asthma | Controls |
| Blood | 32 | 834 | 908 | 832 | 836 | 5.7 | 24.8 |
| (whole) | (21/11) | (705–924) | (698–1033) | (698–900) | (628–995) | (1.0–19.2) | (0.7–42.4) |
| Plasma | 11 | 0.4 | 0.7 | NA | | <LOD | |
| | (5/6) | (0.2–1.14) | (0.4–1.06) | | | | |
| Sputum | 16 | 13.6^{a} | 10.4 | 2.2 | 2.7 | 3.5 | 3.5 |
| (whole) | (10/6) | (10.4–15.6) | (6.6–12.1) | (0.7–8.8) | (1.6–3.5) | (2.2–7.5) | (2.6–4.6) |
| Sputum | 57 | 15.3^{b} | 7.0 | 4.1^{a} | 1.2 | 5.9^{b} | 2.6 |
| (super- | (37/20) | (10.0–22.4) | (4.7–14.3) | (1.4–6.8) | (0.0–3.8) | (4.0–8.4) | (1.8–5.1) |
| natant) | | | | | | | |

| | % GSSG | | GSSG:GSHr Ratio | |
|---|---|---|---|---|
| | Asthma | Controls | Asthma | Controls |
| Blood | 1.7 | 5.9 | 0.01 | 0.03 |
| (whole) | (0.3–5.0) | (0.2–13.8) | (0.00–0.03) | (0.00–0.08) |
| Plasma | NA | | NA | |
| Sputum | 84.2 | 76.7 | 0.92 | 1.65 |
| (whole) | (33.1–93.7) | (63.8–79.8) | (0.20–3.07) | (0.94–1.98) |
| Sputum | 76.6 | 75.2 | 1.49 | 0.67 |
| (supernatant) | (58.1–87.2) | (50.0–100.0) | (0.60–2.57) | (0.44–1.78) |

Data presented as median (quartile 1–quartile 3)
^{a}p < 0.05 versus Controls
^{b}p < 0.005 versus Controls

TABLE 4
Systemic (peripheral blood) and airway (induced sputum) levels of α-tocopherol and
α-tocopherol quinone

| | N | α-tocopherol | | α-tocopherol quinone | | | | α-tocopherol quinone: | |
|---|---|---|---|---|---|---|---|---|---|
| | (Asthma/ | (mg/L) | | (mg/L) | | % α-tocopherol quinone | | α-tocopherol Ratio | |
| | Controls) | Asthma | Controls | Asthma | Controls | Asthma | Controls | Asthma | Controls |
| Blood | 21 | 2.2^{c} | 2.8 | 2.4^{a} | 1.6 | 53.8^{a} | 44.6 | 1.17^{a} | 0.81 |
| (whole) | (9/12) | (1.5–2.8) | (2.1–3.7) | (2.1–3.3) | (1.0–2.5) | (47.2–64.4) | (21.0–51.9) | (0.89–1.82) | (0.27–1.08) |
| Plasma | 22 | 7.3^{a, b} | 12.5 | 0.2 | 0.1 | 2.2 | 1.8 | 0.02 | 0.02 |
| | (9/13) | (5.7–8.1) | (6.6–18.6) | (0.08–0.41) | (0.06–0.59) | (1.4–5.1) | (0.4–4.5) | (0.01–0.05) | (0.00–0.05) |
| Sputum | 19 | 1.7 | 2.3 | 0.15 | 0.12 | 10.8 | 5.7 | 0.12 | 0.06 |
| (whole) | (7/12) | (1.3–2.4) | (1.2–3.0) | (0.09–0.23) | (0.09–0.19) | (5.6–11.8) | (4.0–6.9) | (0.06–0.13) | (0.04–0.07) |

Data presented as median (quartile 1–quartile 3)
^{a}p < 0.05 versus controls;
^{b}Data is parametric and analysed using Student's t-test;
^{c}p = 0.076 versus controls

The results of the present study show differences in the antioxidant status of asthmatics compared to controls, with increased sputum levels of total, reduced and oxidized glutathione, reduced plasma and whole blood levels of (α-tocopherol and increased whole blood levels of (α-tocopherol quinone in asthma, and relate levels of oxidized (α-tocopherol in whole blood (% α-tocopherol quinone) to clinical outcomes (lung function and asthma control). The data also indicates that oxidative stress contributes to epithelial shedding, an important feature of asthma.

Glutathione is present in the lining fluid of the respiratory tract and has a role as both an intra- and extracellular antioxidant. In particular, glutathione directly scavenges free radicals and acts as a co-substrate in the glutathione peroxidase reduction of hydrogen peroxide. α-tocopherol protects lipids that constitute the surfactant lining the lungs that are essential for normal lung function.

Concentrations of α-tocopherol found in sputum in the present study were similar to levels found in peripheral blood. This allocation of α-tocopherol to the lung is evidence that α-tocopherol plays an important role in the lung lining fluid. It has previously been demonstrated that α-tocopherol concentrations in respiratory tract lining fluid (RTLF) increase in response to extreme oxidant burdening of the lungs, probably due to mobilization from other tissues^{[13]}. The levels of α-tocopherol in sputum also indicate the presence of active α-tocopherol-secreting mechanisms that maintain RTLF α-tocopherol levels.

The present study showed low levels of α-tocopherol in the whole blood and plasma of asthmatics compared to healthy controls. This deficiency was not reflected in sputum. This may be a demonstration of movement of α-tocopherol into the airways as an adaptive response to oxidative stress, resulting in a decrease in circulating x-tocopherol levels while airway levels are maintained.

Whole blood concentrations of α-tocopherol are lower than plasma levels. This may be at least partly due to losses to oxidation as demonstrated by the high concentration of α-tocopherol quinone in whole blood. Without wishing to be bound by any one theory, the inventors suggest that these high levels of α-tocopherol quinone may reflect the role of α-tocopherol in protecting erythrocyte membranes from oxidative damage. Sputum ratios of α-tocopherol quinone: α-tocopherol are minimal. Thus, it appears the mechanisms involved in minimising the ratio of oxidized to reduced α-tocopherol in plasma and sputum appear to be working efficiently. No relationship was found between airway and blood antioxidant levels. This is further evidence that active transport mechanisms exist for both α-tocopherol and glutathione whereby airway antioxidant defences are enhanced or maintained in response to a high oxidant load in the lungs. The inverse relationship between whole blood % α-tocopherol quinone and asthma control suggests that a high level of oxidation contributes to a worse clinical outcome, and that clinical status may be improved using therapeutic strategies aimed at reducing oxidative stress.

The results further show that both α-tocopherol and glutathione are important in the respiratory tract. As α-tocopherol quinone is inversely related to clinical status, therapeutic interventions may also be selected to improve the α-tocopherol/α-tocopherol quinone balance to protect against asthma.

### REFERENCES

1. Wood L G, Gibson P G, Garg M L. Biomarkers of lipid peroxidation, airway inflammation and asthma. Eur Resp J 2003; 21:177-186.
2. Kalayci O, Besler T, Kilinc K, Sekerel B E, Saraclar Y. Serum levels of antioxidant vitamins (alpha tocopherol, beta carotene, and ascorbic acid) in children with bronchial asthma. Turkish J Pediatr 2000; 42:17-21.
3. Vural H, Uzun K. Serum and red blood cell antioxidant status in patients with bronchial asthma. Can Respir J 2000; 7:476-480.
4. Shanmugasundaram K R, Kumar S S, Rajajee S. Excessive free radical generation in the blood of children suffering from asthma. Clin Chim Acta 2001; 305:107-114.
5. Kelly F J, Mudway I, Blomberg A, Frew A, Sandstrom T. Altered lung antioxidant status in patients with mild asthma. The Lancet 1999; 354:482-483.
6. Mohan I K, Das U N. Oxidant Stress, antioxidants, nitric oxide and essential fatty acids in bronchial asthma. Med. Sci. Res. 1997; 25:307-309.
7. Smith L J, Houston M, Anderson J. Increased levels of glutathione in bronchoalveolar lavage fluid from patients with asthma. Am Rev Respir Dis 1993; 147:1461-1464.
8. Juniper E F, O'Byrne P M, Guyatt G H, Ferrie P J, King D R. Development and validation of a questionnaire to measure asthma control. Eur Respir J 1999; 14:902-907.
9. Boulet L-P, Boulet V, Milot J. How should we quantify asthma control? Chest 2002; 122:2217-2223.
10. Gibson P G, Wlodarczyk J, Hensley M, Gleeson M, Henry R L, Cripps A W, Clancy R L. Epidemiological association of airway inflammation with asthma symptoms and airway hyperresponsiveness in childhood. Am J Respir Crit Care Med 1998; 158:36-41.
11. Kelly M M, Keatings V, Leigh R, Peterson C, Shute J, Venge P, Djukanovic R. Analysis of fluid-phase mediators. Eur Respir J 2002; 37:24s-39s.
12. Barua A B, Kostic D, Olsen J A. New simplified procedures for the extraction and simultaneous high-performance liquid chromatographic analysis of retinol, tocopherols, and carotenoids in human serum. J Chrom 1993; 617:257-264.
13. Elsayed N M. Antioxidant mobilisation in response to oxidative stress: a dynamic environmental-nutritional interaction. Nutrition 2001; 17:828-834.

1. A method for assessing asthma control in a subject, the method comprising:
(a) obtaining a biological sample from the subject comprising non-lung derived body fluid and/or tissue; and
(b) determining the level of at least one oxidised form of vitamin E or a derivative or metabolite thereof in the non-lung body fluid and/or tissue,
wherein the level of the at least one oxidised form of vitamin E or derivative or metabolite thereof is indicative of the asthma control in the subject.

2. The method of claim 1 wherein the non-lung body fluid and/or tissue is whole blood.

3. The method of claim 1 wherein the oxidised form of vitamin E is α-tocopherol quinone.

4. The method of claim 1 further comprising comparing the level of at least one oxidised form of vitamin E or derivative or metabolite thereof in the sample obtained from the subject with the level of at least one oxidised form of vitamin E or derivative or metabolite thereof from one or more control samples.

5. The method of claim 4 wherein the control sample is a sample from a subject known to have normal levels of the oxidised compound and/or a subject known not to be asthmatic.

6. The method of claim 1 further comprising measuring asthma control in the subject by one or more alternative means and wherein the data so obtained is correlated with the level of the at least one oxidised form of vitamin E or derivative or metabolite thereof.

7. The method of claim 6 wherein the alternative means comprises a questionnaire and/or survey based method.

8. The method of claim 1 further comprising determining the level of vitamin E in the non-lung body fluid and/or tissue and calculating the ratio of the oxidised form to vitamin E, wherein the ratio is indicative of the asthma control in the subject.

9. The method of claim 8 wherein the vitamin E is α-tocopherol.

10. The method of claim 8 further comprising comparing the ratio in the sample obtained from the subject with the ratio from one or more control samples.

11. The method of claim 10 wherein the control sample is a sample from a subject known to have normal levels of the oxidised compound and/or a subject known not to be asthmatic.

12. The method of claim 8 further comprising measuring asthma control in the subject by one or more alternative means and wherein the data so obtained is correlated with the ratio of ratio of oxidised vitamin E to vitamin E.

13. The method of claim 1 comprising the further steps of:
(c) repeating steps (a) and (b) at least once over a period of time; and
(d) determining whether the level has changed over the period of time,
wherein a change in the level is indicative of a change in asthma control in the subject.

14. A method for determining asthma control in a subject, the method comprising:
(a) obtaining a biological sample from the subject comprising non-lung derived body fluid and/or tissue;
(b) determining the level of at least one oxidised form of vitamin E or a derivative or metabolite thereof in non-lung body fluid and/or tissue;
(c) determining the level of vitamin E in the non-lung body fluid and/or tissue; and
(d) calculating the ratio of the level determined in (b) to that determined in (c),
wherein the ratio is indicative of the asthma control in the subject.

15. The method of claim 14 further comprising repeating determining steps (b) and (c) at least once over a period of time; and calculating the ratio of the oxidised form to vitamin E, wherein a change in the ratio is indicative of a change in asthma control in the subject.

16. A method for determining an appropriate asthma treatment regime for a subject, the method comprising:
(a) obtaining a biological sample from the subject comprising non-lung derived body fluid and/or tissue;
(b) determining the level of at least one oxidised form of vitamin E or a derivative or metabolite thereof in the non-lung body fluid and/or tissue;
(c) evaluating the asthma control in the subject on the basis of the level determined in (b); and
(d) selecting a treatment regime for the subject on the basis of the evaluated asthma control.

17. The method of claim 16 further comprising determining the level of vitamin E in the non-lung body fluid and/or tissue, calculating the ratio of the oxidised form to vitamin E and selecting the treatment regime on the basis of the ratio.

18. A method for evaluating the efficacy of an asthma treatment regime in a subject, the method comprising:
(a) treating the subject with a treatment regime for a period sufficient to evaluate the efficacy of the regime;
(b) obtaining a biological sample from the subject comprising non-lung derived body fluid and/or tissue;
(c) determining the level of at least one oxidised form of vitamin E or a derivative or metabolite thereof in the non-lung body fluid and/or tissue;
(d) repeating steps (b) and (c) at least once over a period of time; and
(e) determining whether the level has changed over the period of time,
wherein a change in the level is indicative of a change in asthma control in the subject and the degree of efficacy of the treatment regime.

19. The method of claim 18 further comprising determining the level of vitamin E in the non-lung body fluid and/or tissue more than once over the period of time and calculating the ratio of the oxidised form to vitamin E, wherein a change in the ratio is indicative of a change in asthma control and the degree of efficacy of the treatment regime.

20. A method for diagnosing asthma in a subject, the method comprising:
(a) obtaining a biological sample from the subject comprising non-lung derived body fluid and/or tissue; and
(b) determining the level of at least one oxidised form of vitamin E or a derivative or metabolite thereof in the non-lung body fluid and/or tissue,
wherein the level of the at least one oxidised form of vitamin E or derivative or metabolite thereof is indicative of asthma in the subject.

21. A method for diagnosing asthma in a subject, the method comprising:
(a) obtaining a biological sample from the subject comprising non-lung derived body fluid and/or tissue;
(b) determining the level of at least one oxidised form of vitamin E or a derivative or metabolite thereof in non-lung body fluid and/or tissue;
(c) determining the level of vitamin E in the non-lung body fluid and/or tissue; and
(d) calculating the ratio of the level determined in (b) to that determined in (c), wherein the ratio is indicative of asthma in the subject.
